(19) United States
(12) Patent Application Publication
(10) Pub. No.: US 2010/0016881 A1
(43) Pub. Date: Jan. 21, 2010

(54) BIODEGRADABLE FILTER

(75) Inventors: BRUCE R. FLECK, Anthem, AZ (US); Sean D. Chambers, Bloomington, IN (US); Jacob Lund Clausen, Lyngby (DK); Per Hendriksen, Herlufmagle (DK); Blayne A. Roeder, Lafayette, IN (US)

Correspondence Address: BRINKS HOFER GILSON & LIONE/CHICAGO/COOK, PO BOX 10395, CHICAGO, IL, 60610, US

(73) Assignee: COOK INCORPORATED, Bloomington, IN (US)

(21) Appl. No.: 12/174,160

(22) Filed: Jul. 16, 2008

(51) Int. Cl. A61M 29/00

(52) U.S. Cl. 606/200

(57) ABSTRACT

A medical device that filters thrombi in a body vessel is disclosed. After the need for filtering passes, the device maintains patency in the body vessel without requiring additional steps of percutaneous retrieval or introduction into the patient.

### BACKGROUND OF THE INVENTION

The present invention relates to medical devices. More particularly, the invention relates to a temporary vena cava filter that can be percutaneously placed in the vena cava of a patient and further take on the shape of a stent.

Filtering devices that are percutaneously placed in the vena cava have been available for over thirty years. A need for filtering devices arises in trauma patients, orthopedic surgery patients, neurosurgery patients, or in patients having medical conditions requiring bed rest or non-movement. During such medical conditions, the need for filtering devices arises due to the likelihood of thrombosis in the peripheral vasculature of patients wherein thrombi break away from the vessel wall, risking downstream embolism or embolization. For example, depending on the size, such thrombi pose a serious risk of pulmonary embolism wherein blood clots migrate from the peripheral vasculature through the heart and into the lungs.

A filtering device can be deployed in the vena cava of a patient when, for example, anticoagulant therapy is contraindicated or has failed. Typically, filtering devices are permanent implants, each of which remains implanted in the patient for life, even though the condition or medical problem that required the device has passed. In more recent years, filters have been used or considered in preoperative patients and in patients predisposed to thrombosis which places the patient at risk for pulmonary embolism.

The benefits of a vena cava filter have been well established, but improvements may be made. For example, when the condition that required the filter has passed, there are situations where the body vessel is in need of a stent to maintain the body vessel open or the patency thereof. Retrieval of the filter and percutaneous introduction of a stent would take additional steps to accomplish.

### BRIEF SUMMARY OF THE INVENTION

The present invention provides a medical device that filters thrombi in a body vessel and, after the need for filtering passes, maintains patency in the body vessel without additional steps of percutaneous retrieval or introduction into the patient.

In one embodiment, the present invention provides a filter device for capturing thrombi in a body vessel and transformable to a stent to maintain the body vessel open. The device comprises a plurality of biodegradable threads comprising proximal and distal portions. Each proximal portion has a first end. The first ends are attached together along a longitudinal axis. Each distal portion extends from the proximal portion to a distal end. The distal portions are expandable in the body vessel to engage the body vessel and the first ends are free of contact with the body vessel. Each biodegradable thread is comprised of biodegradable material that degrades at a predetermined time period after the device is deployed in the body vessel.

The device further comprises a radial strut attached to each of the distal ends of the biodegradable threads. The radial strut is radially expandable in the body vessel to engage the distal ends with the body vessel. The radial strut is a z-wire formed into a closed zig-zag configuration including a series of straight sections and a plurality of bends. The straight sections are joined by the bends to form the radial strut.

The device further comprises a biodegradable stabilizer attaching the first ends together and extending radially outwardly to contact the body vessel for balance to the device so that the first ends are free of contact with the body vessel. The biodegradable stabilizer is comprised of bio-absorbable material that degrades at a predetermined time period after the device is deployed in the body vessel.

The device is depressible into a smaller first shape wherein the straight sections are arranged side by side and closely adjacent one another for insertion into the body vessel and the bends store stress therein. The device is expandable, by the release of the stress stored in the bends of the radial strut, into a second shape wherein the straight sections press against the wall of the body vessel. The device is transformable to the stent, by the degradation of the biodegradable stabilizer and threads at the predetermined time period, defining the stent.

In another example, the present invention provides a method for capturing thrombi in a body vessel and for maintaining the body vessel open. The method comprises depressing the device 10 into the first configuration mentioned above. The method further comprises moving the depressed device into a sheath and locating the distal end of the sheath in a blood vessel with the depressed device within the distal end of the sheath. The method further comprises removing the sheath from the blood vessel while holding the stent in place whereby the stress in the second radial strut causes it to expand in the blood vessel to expand the distal portions of the biodegradable threads for engaging the anchoring hooks with the body vessel. The method further comprises degrading the biodegradable stabilizer and threads at the predetermined time period whereby the device takes on a third configuration to maintain the body vessel open.

### BRIEF DESCRIPTION OF THE DRAWINGS

FIG. 1a is a side environmental view a device in an expanded configuration for capturing thrombi and transformable to a stent for maintaining patency of a body vessel in accordance with one embodiment of the present invention;

FIG. 1b is a side environmental view of a device in an expanded configuration in accordance with another embodiment of the present invention;

FIG. 2 is an enlarged view of a radial strut of the device in section 1 of FIG. 1a;

FIG. 3 is an enlarged view of a biodegradable stabilizer of the device in FIG. 1a;

FIG. 4a is a side view of the device in the third configuration for maintaining patency of a blood vessel;

FIG. 4b is a cross-sectional view of the vena cava in which the device is in the third configuration;

FIG. 5 is a side view of the device in a collapsed configuration; and

FIG. 6 is a flow chart of one method for capturing thrombi in a body vessel and maintaining patency of the body vessel in accordance to one example of the present invention.

### DETAILED DESCRIPTION OF THE INVENTION

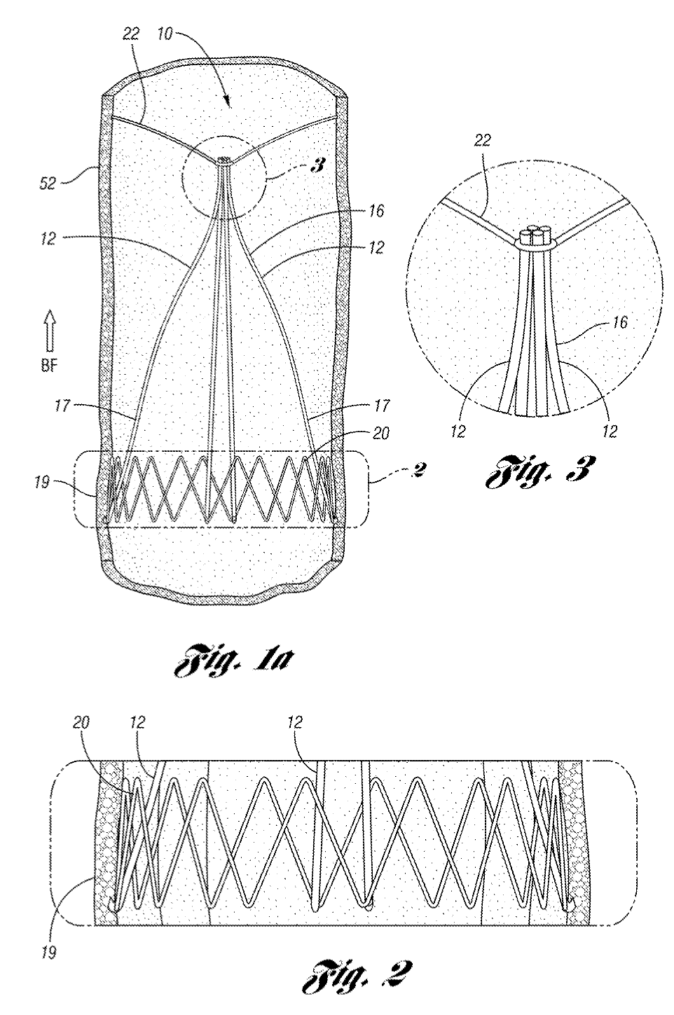

FIG. 1a illustrates a device 10 implanted in a body vessel for capturing thrombi in the body vessel in accordance with one embodiment of the present invention. Preferably, the device 10 is transformable to a stent to maintain patency or openness of the body vessel. As will be described in greater detail below, the device 10 may have a first (compressed) configuration for delivery thereof in a body vessel, a second (expanded) configuration for capturing thrombi in a body vessel, and a third (degraded) configuration for maintaining patency or openness of the body vessel. As shown, the device 10 is in the expanded configuration and comprises a plurality of biodegradable threads 12 having proximal and distal portions 16,17. Each proximal portion 16 has a first end. The first ends 14 are attached together along a longitudinal axis. Each distal portion 17 extends from the proximal portion 16 to a distal end 18 having an anchoring hook. The distal portions are expandable in the body vessel to engage the anchoring hooks 19 with the body vessel. As shown, the first ends 14 are free of contact with the body vessel.

FIG. 1a depicts the device 10 expanded after being deployed in inferior vena cava 52. As shown, the inferior vena cava 52 has been broken away so that the device 10 can be seen. The direction of the blood flow BF is indicated in FIG. 1a by the arrow that is labeled BF. The anchoring hooks 19 17 at the ends of the biodegradable threads 12 are shown as being anchored in the inner lining of the inferior vena cava 52. The anchoring hooks 19 17 include barbs 29 that, in one embodiment, project toward the hub 11 of the filter. The barbs 29 function to retain the device 10 in the location of deployment.

The spring biased configuration of the radial strut 20 causes the anchoring hooks 19 to engage the vessel wall and anchor the device at the location of deployment. After initial deployment, the pressure of the blood flow on the device 10 contributes in maintaining the barbs 29 anchored in the inner lining of the inferior vena cava 52.

Each biodegradable thread 12 is comprised of biodegradable material that degrades at a predetermined time period after the device 10 is deployed in the body vessel. Preferably, the biodegradable thread 12 is a biodegradable suture wire that may be made of any suitable material, such as polylactide or polyglycolide. The predetermined time period may be any suitable time period for the device 10 to effectively filter thrombi in the body vessel and begin to maintain the patency of the body vessel. For example, the time period may be between about two to ten weeks, preferably between about three to six weeks. However, any other time period may be acceptable without falling beyond the scope or spirit of the present invention.

As shown in FIG. 1a, the device 10 further comprises a radial strut 20 attached to each of the distal ends 18 of the biodegradable threads 12. The radial strut 20 is disposed about the distal portions of the biodegradable threads 12 and is expandable therewith in the body vessel. The radial strut 20 is radially expandable in the body vessel to engage the anchoring hooks 19 of the distal ends 18 with the body vessel. Preferably, the radial strut 20 is formed from a superelastic material, stainless steel wire, Nitinol, cobalt-chromium-nickel-molybdenum-iron alloy, or cobalt chrome-alloy, or any other suitable material that will result in a self-opening or self-expanding device 10.

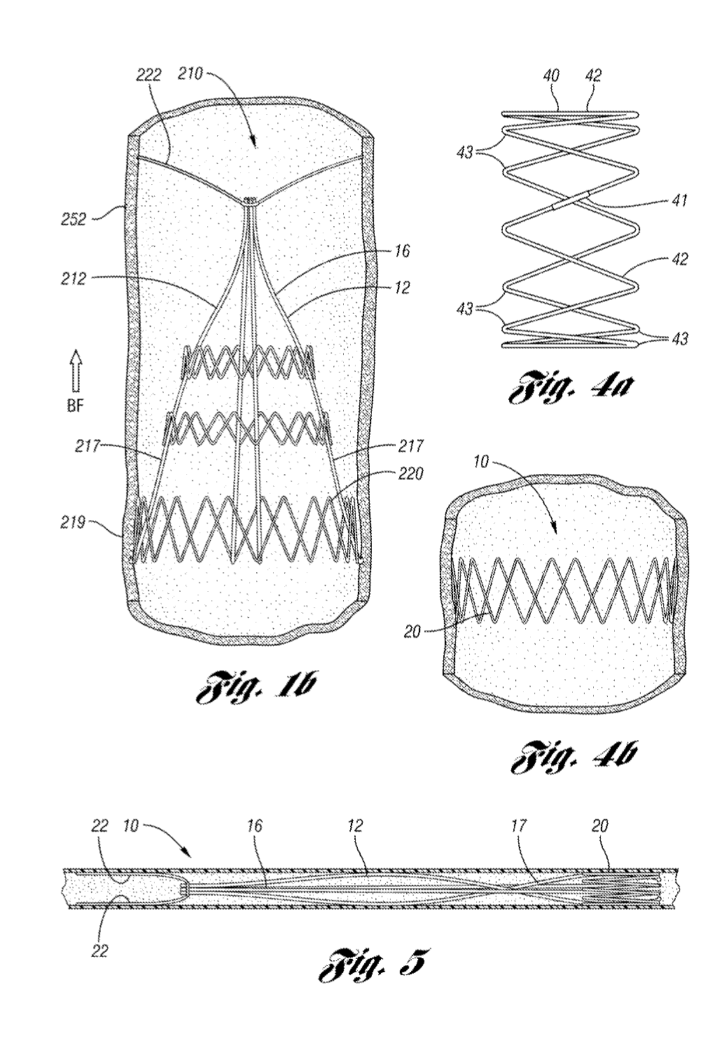

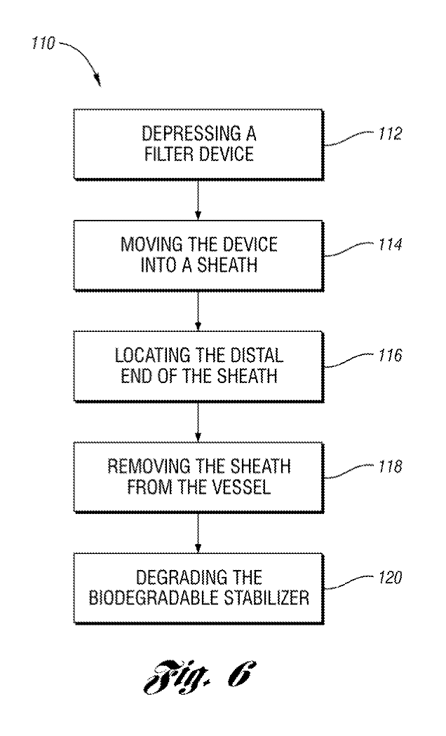

The radial strut 20 is preferably a z-wire formed into a closed zig-zag configuration including a series of straight sections and a plurality of bends. The straight sections are joined by the bends to form the radial strut. Of course, the radial strut 20 may be comprised a plurality of z-wires without falling beyond the scope or spirit of the present invention. As shown in FIGS. 2 and 4a-4b, the radial strut 20 includes a length 40 of wire formed in a closed zig-zag configuration. The wire is closed by a sleeve 41 which is welded to or tightly squeezed against the ends of the wire to produce the endless configuration. Referring to FIG. 5, the radial strut 20 is shown in a resiliently compressed first configuration wherein the straight sections 42 are arranged side-by-side and closely adjacent one another. The straight sections of the stent are joined by bends 43 which are relatively sharp.

Referring to FIG. 1a, the device 10 further comprises a biodegradable stabilizer 22 that attaches the first ends 14 together in a closed position. In this embodiment, the biodegradable stabilizer 22 is a biodegradable thread 12 having a pair of contact ends. As shown, the biodegradable stabilizer 22 ties the first ends 14 together and extends radially outwardly to contact the contact ends with body vessel for balance to the device 10. Thus, the biodegradable stabilizer 22 is preferably configured or made of material more rigid than the biodegradable threads 12. This may be accomplished by diameter size or material or both. This allows the first ends 14 to be free of contact with the body vessel. The biodegradable stabilizer 22 is thus comprised of bio-absorbable material that is degrades at a predetermined time period after the device 10 is deployed in the body vessel. Preferably, the biodegradable stabilizer 22 is made of any suitable material, such as polylactide or polyglycolide.

FIG. 1b illustrates a device 80 for capturing thrombi in a body vessel and further maintaining patency in the body vessel. As shown, the device 10 comprises similar components to the device 10 in FIG. 1a such as the biodegradable threads 82, first ends 84 and stabilizer 85. However, the device 80 further includes a plurality of radial struts 86 connected about the biodegradable threads and spaced apart from each other along the longitudinal axis of the device 80. As shown, the radial struts 86 are longitudinally spaced apart from each other. The placement of the radial struts provides support to the body vessel to maintain a relatively larger area of a body vessel open where needed.

As mentioned above, the device 10 is depressible into a collapsed or first configuration (shape) as shown in FIG. 5. As such, the straight sections are arranged side by side and closely adjacent one another for insertion into the body vessel and the bends store stress therein. The device 10 is expandable, by the release of the stress stored in the bends of the radial strut, into an expanded or second configuration wherein the straight sections press against the wall of the body vessel. After degradation of the biodegradable threads 12 and the stabilizer 22, the device 10 is take on a third (degraded) configuration having the shape of a stent. This is accomplished by the degradation of the biodegradable stabilizer 22 and threads 12 at the predetermined time period, defining the stent. In one embodiment, the radial strut 20 may be comprised of one or a plurality of z-wires connected about the biodegradable threads 12 and spaced apart from each other along the longitudinal axis of the device 10 as mentioned above and shown in FIG. 1b.

FIG. 4b illustrates the device 10 in the third configuration implanted in vena cava 50. As shown, the device 10 takes on the shape of a stent for maintaining patency of the vena cava degradation of the biodegradable member. As mentioned, the biodegradable threads 12 and stabilizer 22 defines the second configuration (FIG. 1a) of the device 10 and degrades after deployment in the body vessel. Upon degradation, the device 10 transforms to the third configuration mentioned above and takes on the shape of a stent to maintain patency of the body vessel.

FIG. 3b illustrates a cross-sectional view of the device 10 of FIG. 3a at hub area 11. As shown, the hub area 11 is formed of a bundle of first ends 14 of the longitudinal threads 12 attached together by the biodegradable stabilizer 22. The threads 12 may be attached together by the biodegradable stabilizer 22 by any suitable manner such as by tying, sonic welding, or biodegradable adhesives.

FIG. 6 illustrates one method 110 for capturing thrombi in a body vessel and for maintaining the body vessel open in accordance with one example of the present invention. As shown, the method comprises depressing the device 10 to the first configuration in box 112 and introducing or “loading” the device 10 into a delivery catheter in box 114. For deployment of the device 10, the delivery tube is percutaneously inserted through the patient's vessel such that the distal end 18 of the delivery tube is at the location of deployment. In this embodiment, a wire guide is preferably used to guide the delivery tube to the location of deployment.

The method further comprises locating the distal end 18 of the sheath in a body vessel in box 116 wherein the device 10 in the first configuration is disposed within the distal end 18 of the sheath. The method further comprises removing the sheath from the body vessel while holding the device 10 in place in box 118. The stress in the radial strut 20 causes it to expand the distal portions of the biodegradable threads 12 to the second configuration for engaging the anchoring hooks 19 with the body vessel.

When the device 10 is expanded in the vena cava, the anchoring hooks 19 17 of the biodegradable threads 12 are in engagement with the vessel wall. The anchoring hooks 19 17 of the biodegradable threads 12 have anchored the device 10 at the location of deployment in the vessel, preventing the device 10 from moving with the blood flow through the vessel. The method further comprises degrading the biodegradable threads 12 and the stabilizer 22 at the predetermined time period in box 120 whereby the device 10 takes on a shape of a stent in the third configuration to maintain the body vessel open.

While the present invention has been described in terms of preferred embodiments, it will be understood, of course, that the invention is not limited thereto since modifications may be made to those skilled in the art, particularly in light of the foregoing teachings.

What is claimed is:

1. A filter device for capturing thrombi in a body vessel and transformable to a stent to maintain the body vessel open, the filter comprising:
a plurality of biodegradable threads comprising proximal and distal portions, each proximal portion having a first end, the first ends attached together along a longitudinal axis, each distal portion extending from the proximal portion to a distal end, the distal portions being expandable in the body vessel to engage the body vessel and the first ends being free of contact with the body vessel, each biodegradable thread being comprised of biodegradable material that degrades at a predetermined time period after the device is deployed in the body vessel;
a radial strut attached to each of the distal ends of the biodegradable threads, the radial strut being radially expandable in the body vessel to engage the distal ends with the body vessel, the radial strut being a z-wire formed into a closed zig-zag configuration including a series of straight sections and a plurality of bends, the straight sections being joined by the bends to form the radial strut; and
a biodegradable stabilizer attaching the first ends together and extending radially outwardly to contact the body vessel for balance to the device so that the first ends are free of contact with the body vessel, the biodegradable stabilizer being comprised of bio-absorbable material that degrades at a predetermined time period after the device is deployed in the body vessel;
wherein the device is depressible into a smaller first shape wherein the straight sections are arranged side by side and closely adjacent one another for insertion into the body vessel and the bends store stress therein,
wherein the device is expandable, by the release of the stress stored in the bends of the radial strut, into a second shape wherein the straight sections press against the wall of the body vessel; and
wherein the device is transformable to the stent, by the degradation of the biodegradable stabilizer and threads at the predetermined time period, defining the stent.

2. The device of claim 1 wherein the biodegradable stabilizer is a biodegradable suture wire.

3. The device of claim 1 wherein the radial strut comprises a plurality of z-wires, each wire being disposed to the biodegradable threads and longitudinally spaced apart from each other.

4. The device of claim 1 wherein the predetermined time period is between about 2 and 10 weeks.

5. The device of claim 1 wherein the biodegradable thread is made of polylactide or polyglycolide.

6. The device of claim 1 wherein the biodegradable stabilizer is made of polylactide or polyglycolide.

7. A filter device for capturing thrombi in a body vessel and transformable to a stent, the filter comprising:
a plurality of biodegradable threads comprising proximal and distal portions, each proximal portion having a first end, the first ends attached together along a longitudinal axis, each distal portion extending from the proximal portion to a distal end, the distal portions being expandable in the body vessel to engage the body vessel and the first ends being free of contact with the body vessel, each biodegradable thread being comprised of biodegradable material that degrades at a predetermined time period after the device is deployed in the body vessel;
a radial strut attached to each of the distal ends of the biodegradable threads, the radial strut being radially expandable in the body vessel to engage the distal ends with the body vessel, the radial strut being a z-wire formed into a closed zig-zag configuration including a series of straight sections and a plurality of bends, the straight sections being joined by the bends to form the radial strut; and
a biodegradable stabilizer attaching the first ends together and extending radially outwardly to contact the body vessel for balance to the device so that the first ends are free of contact with the body vessel, the biodegradable stabilizer being comprised of bio-absorbable material that degrades at a predetermined time period after the device is deployed in the body vessel.

8. The device of claim 7 wherein the biodegradable stabilizer is a biodegradable suture wire.

9. The device of claim 7 wherein the radial strut comprises a plurality of z-wires, each wire being disposed to the biodegradable threads and longitudinally spaced apart from each other.

10. The device of claim 7 wherein the predetermined time period is between about 2 and 10 weeks.

11. The device of claim 7 wherein the biodegradable thread is made of polylactide or polyglycolide.

12. The device of claim 7 wherein the biodegradable stabilizer is made of polylactide or polyglycolide.

13. A method for capturing thrombi in a body vessel and for maintaining the body vessel open, the method comprising:
depressing a filter device into a first shape, the filter device comprising;
a plurality of biodegradable threads comprising proximal and distal portions, each proximal portion having a first end, the first ends attached together along a longitudinal axis, each distal portion extending from the proximal portion to a distal end, the distal portions being expandable in the body vessel to engage the body vessel and the first ends being free of contact with the body vessel, each biodegradable thread being comprised of biodegradable material that degrades at a predetermined time period after the device is deployed in the body vessel;
a radial strut attached to each of the distal ends of the biodegradable threads, the radial strut being radially expandable in the body vessel to engage the distal ends with the body vessel, the radial strut being a z-wire formed into a closed zig-zag configuration including a series of straight sections and a plurality of bends, the straight sections being joined by the bends to form the radial strut; and
a biodegradable stabilizer attaching the first ends together and extending radially outwardly to contact the body vessel for balance to the device so that the first ends are free of contact with the body vessel, the biodegradable stabilizer being comprised of bio-absorbable material that degrades at a predetermined time period after the device is deployed in the body vessel;
moving the depressed device into a sheath;
locating the distal end of the sheath in a blood vessel with the depressed device within the distal end of the sheath;
removing the sheath from the blood vessel while holding the stent in place whereby the stress in the second radial strut causes it to expand in the blood vessel to expand the distal portions of the biodegradable threads for engaging the anchoring hooks with the body vessel; and
degrading the biodegradable member at the predetermined time period whereby the stress in the first radial strut is released into a third shape to maintain the body vessel open.

14. The method of claim 13 wherein the biodegradable stabilizer is a biodegradable suture wire.

15. The method of claim 13 wherein the radial strut comprises a plurality of z-wires, each wire being disposed to the biodegradable threads and longitudinally spaced apart from each other.

16. The method of claim 13 wherein the predetermined time period is between about 2 and 10 weeks.

17. The method of claim 13 wherein the biodegradable thread is made of polylactide or polyglycolide.

18. The method of claim 13 wherein the biodegradable stabilizer is made of polylactide or polyglycolide.
